(19) United States
(12) Patent Application Publication
(10) Pub. No.: US 2024/0392330 A1
(43) Pub. Date: Nov. 28, 2024

(54) PRETREATMENT OF ORGANIC WASTE FOR LACTIC ACID PRODUCTION EMPLOYING COMPOSITIONS OF BACILLUS COAGULANS SPORES

(71) Applicant: TRIPLEW LTD., Netanya (IL)

(72) Inventors: Ofir Avidan, Lod (IL); Tsvika Greener, Ness Ziona (IL)

(21) Appl. No.: 18/695,807

(22) PCT Filed: Sep. 29, 2022
(86) PCT No.: PCT/IL2022/051034

§ 371 Date: Mar. 26, 2024

Related U.S. Application Data
(60) Provisional application No. 63/249,684, filed on Sep. 29, 2021.

(51) Int. Cl. C12P 7/56; B09B 3/60; B09B 101/70; C12N 1/20

(52) U.S. Cl. CPC C12P 7/56; B09B 3/60; C12N 1/20; B09B 2101/70

(57) ABSTRACT

Methods and systems for pretreatment of organic waste prior to large-scale production of lactic acid from the organic waste are provided, which employ dried or partially-dried compositions of Bacillus coagulans spores. The methods and systems advantageously provide partial conversion of the organic waste to L-lactic acid already at the pretreatment stage, thereby producing an improved feedstock material for large-scale production of L-lactic acid.

### FIELD OF THE INVENTION

The present invention relates to methods for pretreatment of organic waste prior to large-scale production of lactic acid from the organic waste, which employ dried or partially dried compositions of Bacillus coagulans spores. The methods of the present invention advantageously provide partial conversion of the organic waste to L-lactic acid already at the pretreatment stage, which lowers the pH of the organic waste such that microorganisms producing undesired products are inhibited while L-lactic acid is enriched. The present invention thereby provides an improved feedstock material for large-scale production of L-lactic acid.

### BACKGROUND OF THE INVENTION

Lactic acid fermentation, namely, production of lactic acid from carbohydrate sources via microbial fermentation, has been gaining interest in recent years due to the ability to use lactic acid as a building block in the manufacture of bioplastics. Lactic acid can be polymerized to form the biodegradable and recyclable polyester polylactic acid (PLA), which is considered a potential substitute for plastics manufactured from petroleum. PLA is used in the manufacture of various products including food packaging, disposables, fibers in the textile and hygiene products industries, and more. PLA is the most widely used plastic filament material in 3D printing.

Production of lactic acid by fermentation bioprocesses is preferred over chemical synthesis methods for various considerations, including environmental concerns, costs, and the difficulty to generate enantiomerically pure lactic acid by chemical synthesis, which is desired for most industrial applications of PLA. The conventional fermentation process is typically based on anaerobic fermentation by lactic acid-producing microorganisms, which produce lactic acid as the major metabolic end product of carbohydrate fermentation. For production of PLA, the lactic acid generated during the fermentation is separated from the fermentation broth and purified by various downstream processes, and the purified lactic acid is then subjected to polymerization.

Lactic acid has a chiral carbon atom and therefore exists in two enantiomeric forms, D- and L-lactic acid. In order to generate PLA that is suitable for industrial applications, the polymerization process should utilize only one enantiomer. Presence of impurities or a racemic mixture of D- and L-lactic acid results in a polymer having undesired characteristics such as low crystallinity and low melting temperature. Thus, lactic acid microorganisms that produce only L-lactate enantiomer or only D-lactate enantiomer are typically used.

In currently available commercial processes, the carbohydrate source for lactic acid fermentation is typically a starch-containing renewable source such as corn and cassava root. Additional sources, such as the cellulose-rich sugarcane bagasse, have also been proposed.

An additional source of carbohydrates for lactic acid fermentation that has been proposed is complex organic waste, such as mixed food waste from municipal, industrial, and commercial origin. Such organic waste is advantageous as it is readily available and less expensive compared to other carbohydrate sources for lactic acid fermentation. However, the conversion of complex organic wastes to useful fermentation products such as lactic acid on an industrial scale faces numerous technical challenges and requires precise control over operational conditions, including pretreatment, pH, temperature, microbes and more. Improvements are needed in order to make the process economically feasible on an industrial scale.

Sakai et al. (2012), Total Recycle System of Food Waste for Poly-L-Lactic Acid Output, Advances in Applied Biotechnology, Marian Petre, IntechOpen, describes a total recycle system of food waste using B. coagulans fermentation.

U.S. Pat. No. 9,376,697 discloses a method for on-farm processing a biomass feedstock into a useful industrial chemicals includes the steps of (a) delignifying the biomass feedstock to produce a delignified biomass, (b) subjecting the delignified biomass to cellulase production, (c) subjecting the delignified biomass with attached cellulase to simultaneous cellulolytic and solventogenic reactions to produce useful industrial chemicals (d) collecting and separating the useful industrial chemical from the fermentation broth and (e) collecting the fermentation residues.

WO 2017/122197, assigned to the Applicant of the present invention, discloses dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides.

WO 2020/208635, assigned to the Applicant of the present invention, discloses systems and methods for processing organic waste, particularly mixed food waste, using a D-lactate oxidase. The D-lactate oxidase eliminates D-lactic acid that is present in the organic waste. The processed organic waste can be used as a substrate in industrial fermentation processes, such as production of optically-pure L-lactic acid.

WO 2021/191901, assigned to the Applicant of the present invention, discloses systems and methods for recycling of organic waste to produce lactic acid by fermentation, which utilize dried or partially-dried compositions of spores of the lactic acid-producing bacterium Bacillus coagulans.

There remains a need to improve the production of lactic acid from organic waste on an industrial scale, in order to make the process more economically feasible. It would be highly advantageous to have systems and methods that simplify the process, reduce costs and improve the overall yield.

### SUMMARY OF THE INVENTION

The present invention provides methods and systems for pretreatment of organic waste prior to large-scale production of lactic acid from the organic waste, which employ dried or partially-dried compositions of spores of homofermentative, spore-forming lactic acid-producing bacteria, e.g., Bacillus sp., in particular, Bacillus coagulans spores. The methods and systems of the present invention advantageously provide partial conversion of the organic waste to L-lactic acid already at the pretreatment stage, thereby producing an improved feedstock material for large-scale production of L-lactic acid.

In particular embodiments, the organic waste is mixed food waste, municipal waste and agricultural waste. As disclosed herein, dried or partially-dried compositions of Bacillus coagulans spores may be mixed with the organic waste in garbage bins, dumpsters, waste handling containers, waste management vehicles, garbage trucks, waste capture equipment and the like, or during processing of the organic waste prior to fermentation at organic waste management facilities, for example, during grinding, mincing and/or shredding of the organic waste to reduce its particle size. The spores can germinate under a broad range of conditions. Germination occurs when suitable conditions are formed, and vegetative cells that germinate begin the conversion of the organic waste (in particular, conversion of soluble reducing sugars present in the organic waste) into L-lactic acid. The addition of the spores and production of L-lactic acid already at the pretreatment stage is advantageous, for example, for: controlling natural decay processes while the organic waste is being processed and transported to a fermenter, potentially preventing other competing fermentation processes to occur; increasing the L/D ratio by controlling the natural decay; controlling fermentation lag time and shortening fermentation lag time; and acidifying the organic waste, thereby reducing other competing microorganism load. As exemplified hereinbelow, spore compositions as disclosed herein were able to germinate and acidify waste and growth media of various compositions, under various temperatures and growth conditions.

Organic waste of the present invention comprises solid and non-solid materials at varying ratios, depending on its source. For example, fruit and vegetable waste typically includes a high percentage of water, up to 95% water (w/w), e.g., 90%-95% water (w/w), while bakery waste includes a lower percentage of water, up to 50% (w/w). Mixed food waste, containing food waste of different sources, may include 30%-90% water, for example, 40%-80%, 45%-75%, including each value within the range. Each possibility represents a separate embodiment of the present invention.

The present invention further discloses large-scale production of L-lactic acid using dried or partially-dried compositions of B. coagulans spores, wherein a B. coagulans spore composition is suspended in an acidic solution of an organic acid, particularly a lactic acid solution, prior to addition of the composition into a lactic acid production fermenter. In some embodiments, the solution is a buffered solution comprising a conjugate base of lactic acid, in addition to lactic acid, e.g., magnesium lactate. As disclosed herein, the spores survive suspension in a lactic acid solution and successfully germinate following such treatment, thus providing simple means for inactivating microbial contaminants that may be present in the composition, prior to inoculation into the production fermenter.

In some embodiments, the concentration of lactic acid in the solution is in the range of 0.5%-5%, with pH in the range of 2-4, including each value within the ranges. In some embodiments, the concentration of lactic acid in the solution is in the range of 1%-5%, or in the range of 2%-4%, including each value within the range. Each possibility represents a separate embodiment. In some embodiments, the pH of the solution is in the range of 2.5-4, including each value within the range. Exemplary concentrations of lactic acid include 3%, with pH 3.5.

Suspension in the acidic solution, particularly lactic acid solution, may be carried out for a few minutes up to several hours. Preferably, the suspending in a lactic acid solution comprises incubating the suspension for between 5 min to 1 hr (preferably between 5 min to 45 min) at a temperature between ambient temperature up to 50° C., for example, between 25-45° C.

Compositions of dried or partially-dried spores as disclosed herein can be easily transported to organic waste collection points or organic waste management sites, stored and removed from storage upon need. The spores in the dried or partially-dried compositions can successfully recover from storage, germinate and ferment organic waste to lactic acid at high yields. Advantageously, the dried or partially-dried 15 compositions of spores do not require cooling and sustain various storage conditions for prolonged periods of time. Viability of the spores is maintained throughout storage, and cell loss following drying and storage is minimal.

The utilization of organic waste as a substrate for fermentation as described herein is highly advantageous compared to previously described lactic acid production processes which utilize source materials that are of high value as human food.

A dried composition of spores as disclosed is characterized by moisture content of up to 15% (w/w) or any amount therebetween. In some embodiments, the dried composition of B. coagulans spores is characterized by moisture content of up to 10% (w/w). In some embodiments, the dried composition of B. coagulans spores is characterized by moisture content of 4%-15% (w/w), for example 4%-10% (w/w). Each possibility represents a separate embodiment of the present invention.

A partially-dried composition of spores as disclosed herein is characterized by a moisture content in the range of 15%-30% (w/w) or any amount therebetween. In some embodiments, the partially-dried composition of B. coagulans spores is characterized by a moisture content in the range of 15%-25% (w/w) or any amount therebetween.

As provided herein, the moisture content of a dried or semi-dried inoculum, formulation or composition comprising B. coagulans spores refers to the amount of water outside the spores (namely, “moisture content” as used herein does not include water found inside the spores). The moisture content is provided as a percentage out of the total weight of the inoculum, formulation or composition. The terms “inoculum”, “formulation” and “composition” of spores are used herein interchangeably to describe a composition containing the spores, wherein the composition may be dried or semi-dried.

According to one aspect, the present invention provides a method for preparing a feedstock material for large-scale production of L-lactic acid or a salt thereof, the method comprising:
(i) providing an unprocessed organic waste;
(ii) mixing the unprocessed organic waste with a dried or partially-dried composition of spores of the L-lactic acid producer Bacillus coagulans; and
(iii) subjecting the unprocessed organic waste to one or more of mechanical treatment, chemical treatment and enzymatic treatment;
wherein the mixing of step (ii) is carried out prior to, and/or during, the one or more treatments of step (iii),
wherein preparing the feedstock material is carried out under non-sterile conditions, and
wherein germination of at least a portion of the B. coagulans spores occurs during the preparation of the feedstock material such that the organic waste is partially converted into L-lactic acid,
thereby obtaining a feedstock material enriched with L-lactic acid for large-scale production of L-lactic acid.

In some embodiments, the organic waste is selected from the group consisting of food waste, municipal waste, agricultural waste, plant material and a mixture or combination thereof. Each possibility represents a separate embodiment of the present invention.

In some embodiments, the organic waste is a solid organic waste.

In some embodiments, the organic waste is a semi-solid organic waste with a water content in the range of 30%-95% (w/w).

In some embodiments, the organic waste is a liquid organic waste.

In some embodiments, the mixing in step (ii) is carried out in organic waste collection containers.

In some embodiments, the mixing in step (ii) is carried out in transport vehicles that transport organic waste to waste management facilities.

In some embodiments, the mixing in step (ii) is carried out at an organic waste management facility prior to, and/or during, the one or more treatments of step (iii).

In some embodiments, the one or more treatments of step (iii) comprise size reduction by mincing, shredding, grinding or a combination thereof.

In some embodiments, the one or more treatments of step (iii) comprise saccharification using one or more polysaccharide-degrading enzyme.

In some embodiments, step (ii) further comprises mixing with one or more polysaccharide-degrading enzyme.

According to a further aspect, the present invention provides a method for reducing microorganism load in organic waste and enriching for L-lactic acid prior to large-scale production of L-lactic acid, the method comprising:
(i) providing an unprocessed organic waste;
(ii) mixing the unprocessed organic waste with a dried or partially-dried composition of spores of the L-lactic acid producer Bacillus coagulans; and
(iii) subjecting the unprocessed organic waste to one or more of mechanical treatment, chemical treatment and enzymatic treatment;
wherein the mixing of step (ii) is carried out prior to, and/or during, the one or more treatments of step (iii),
wherein the reduction of microorganism load and enrichment for L-lactic acid is carried out without the addition of a pH-adjusting agent and under non-sterile conditions, and
wherein germination of at least a portion of the B. coagulans spores occurs during the processing of the organic waste such that the organic waste is partially converted into L-lactic acid,
thereby obtaining a decrease of pH reducing the load of microorganisms endogenously present in the organic waste, and an enrichment of L-lactic acid.

According to a further aspect, the present invention provides a method for large-scale production of L-lactic acid, the method comprising:
(a) preparing a feedstock material as disclosed herein;
(b) optionally mixing the feedstock material with additional B. coagulans spores; and
(c) incubating the feedstock material in a fermentation reactor under controlled conditions for lactic acid production by Bacillus coagulans, to thereby produce L-lactic acid.

In some embodiments, the feedstock is not sterilized (e.g., using high pressure steam or using chemicals) prior to the incubation in the fermentation reactor, and the large-scale production is an open-fermentation carried out under non-sterile conditions.

In other embodiments, the feedstock is sterilized prior to the incubation in the fermentation reactor (e.g., using a steam jet-cooker), and the method further comprises mixing the feedstock material with additional B. coagulans spores following sterilization.

In some embodiments, step (c) comprises incubating the feedstock material in a fermentation reactor with one or more polysaccharide-degrading enzyme under controlled conditions for degradation of polysaccharides by the one or more polysaccharide-degrading enzyme and production of lactic acid by the B. coagulans.

In some embodiments, the composition of B. coagulans spores is a dried composition characterized by moisture content of less or equal to 10% w/w.

In some embodiments, the composition of B. coagulans spores is a dried composition comprising magnesium lactate.

In some embodiments, the dried composition of B. coagulans spores comprises 10\hat{ }8-10\hat{ }10 spores/g powder, and wherein the concentration of the magnesium lactate in the dried composition is in the range of 40-60% (w/w).

According to a further aspect, the present invention provides a feedstock material for large-scale production of L-lactic acid, comprising:
(a) semi-solid processed organic waste comprising 5%-30% solids (w/w) (preferably 5%-20% solids (w/w)); and
(b) B. coagulans bacterial cells, B. coagulans spores or a combination thereof,
wherein the feedstock product is enriched with L-lactic acid compared to D-lactic acid, and
wherein the pH of the feedstock product is in the range of 3-6, including each value within the range. In some embodiments, the pH of the feedstock product is in the range of 3-5, including each value within the range. In some embodiments, the pH of the feedstock product is in the range of 4-5, including each value within the range.

According to a further aspect, the present invention provides a method for preparing a feedstock material for large-scale production of L-lactic acid or a salt thereof, the method comprising:
(i) providing an unprocessed organic waste;
(ii) mixing the unprocessed organic waste with a dried or partially-dried composition of spores of a spore-forming L-lactic acid producing Bacillus sp.; and
(iii) subjecting the unprocessed organic waste to one or more of mechanical treatment, chemical treatment and enzymatic treatment,
wherein the mixing of step (ii) is carried out prior to, and/or during, the one or more treatments of step (iii),
wherein preparing the feedstock material is carried out under non-sterile conditions, and
wherein germination of at least a portion of the Bacillus spores occurs during the preparation of the feedstock material such that the organic waste is partially converted into L-lactic acid,
thereby obtaining a feedstock material enriched with L-lactic acid for large-scale production of L-lactic acid.

According to a further aspect, the present invention provides a method for reducing microorganism load in organic waste and enriching for L-lactic acid prior to large-scale production of L-lactic acid, the method comprising:
(i) providing an unprocessed organic waste;
(ii) mixing the unprocessed organic waste with a dried composition of spores of a spore-forming L-lactic acid producing Bacillus sp.; and
(iii) subjecting the unprocessed organic waste to one or more of mechanical treatment, chemical treatment and enzymatic treatment;
wherein the mixing of step (ii) is carried out prior to, and/or during, the one or more treatments of step (iii),
wherein the reduction of microorganism load and enrichment for L-lactic acid is carried out without the addition of a pH-adjusting agent and under non-sterile conditions, and
wherein germination of at least a portion of the Bacillus spores is induced during the processing of the organic waste such that the organic waste is partially converted into L-lactic acid,
thereby obtaining a decrease of pH reducing the load of microorganisms endogenously present in the organic waste, and an enrichment of L-lactic acid.

According to a further aspect, the present invention provides a method for large-scale production of L-lactic acid, the method comprising:
(a) preparing a feedstock material as disclosed herein;
(b) optionally mixing the feedstock material with additional Bacillus spores; and
(c) incubating the feedstock material in a fermentation reactor under controlled conditions for lactic acid production by the Bacillus sp., to thereby produce L-lactic acid.

In some embodiments, the Bacillus sp. is selected from the group consisting of Bacillus stearothermophilus, Bacillus licheniformis, Bacillus subtilis, Bacillus laevolacticus, Bacillus racemilacticus, Bacillus thermoamylovorans, Sporolactobacillus, Sporolactobacillus shoreicorticis, Sporolactobacillus vineae, Sporolactobacillus nakayamae, Terrilactibacillus laevilacticus, and Terrilactibacillus tamarindi. Each possibility represents a separate embodiment of the present invention.

According to a further aspect, the present invention provides a method for recycling organic waste to produce lactic acid or a salt thereof, the method comprising:
(I) providing a pretreated organic waste that was subjected to pretreatment comprising reduction of particle size and optionally sterilization;
(II) providing a dried or partially-dried composition of B. coagulans spores;
(III) suspending the dried or partially-dried composition of B. coagulans spores in a lactic acid solution, to obtain a B. coagulans spore suspension in which microbial contaminants are inactivated; and
(IV) mixing the pretreated organic waste in a fermentation reactor with one or more saccharide-degrading enzyme and the B. coagulans spore suspension, and incubating the mixture in the fermentation reactor to saccharify the organic waste and induce germination of the spores and subsequently lactic acid production by vegetative B. coagulans cells that germinate from the spores; and
(V) recovering lactic acid or a salt thereof from the fermentation broth.

In some embodiments, the incubating in step (IV) is carried out at a pH in the range of 5-7.

In some embodiments, the incubating in step (IV) is carried out at a pH in the range of 5.5-6.5.

In some embodiments, the incubating in step (IV) is carried out at a temperature in the range of 45-60° C.

In some embodiments, the incubating in step (IV) is carried out at a temperature in the range of 50-55° C.

In some embodiments, the incubating in step (IV) is carried out for a period of time in the range of 20-48 hours.

In some embodiments, the incubating in step (IV) is carried out for a period of time in the range of 20-36 hours.

In some embodiments, the one or more saccharide-degrading enzyme is a polysaccharide-degrading enzyme selected from the group consisting of an amylase, a cellulase and a hemicellulase.

In some embodiments, the one or more saccharide-degrading enzyme comprises a glucoamylase.

In some embodiments, the mixing in step (IV) comprises adding the dried composition of B. coagulans to the fermentation reactor to obtain at least 10\hat{ }4 spores/ml fermentation medium.

In some embodiments, the mixing in step (IV) comprises adding the dried composition of B. coagulans to the fermentation reactor to obtain at least 10\hat{ }6 spores/ml fermentation medium.

Other objects, features and advantages of the present invention will become clear from the following description and examples.

### BRIEF DESCRIPTION OF THE FIGURES

FIG. 1. Illustration of seeding dried or partially-dried compositions of Bacillus coagulans spores (“BC spores”) according to some embodiments of the present invention.

### DETAILED DESCRIPTION OF THE INVENTION

The present invention is directed to industrial fermentation processes for production of lactic acid from organic waste, in which dried or semi-dried (partially-dried) compositions of Bacillus coagulans spores are used.

In some embodiments, there is provided herein a method for preparing a feedstock material for large-scale production of lactic acid or a salt thereof, the method comprising: (i) providing an unprocessed organic waste; (ii) mixing the unprocessed organic waste with a dried composition of spores of the L-lactic acid producer Bacillus coagulans; and (iii) subjecting the unprocessed organic waste to one or more of mechanical treatment, chemical treatment and enzymatic treatment, wherein the mixing of step (ii) is carried out prior to, or during, the one or more treatments of step (iii), wherein preparing the feedstock material is carried out under non-sterile conditions, and wherein germination of at least a portion of the B. coagulans spores is induced during the preparation of the feedstock material such that the organic waste is partially converted into L-lactic acid, thereby obtaining a feedstock material for large-scale production of lactic acid.

In some embodiments, there is provided herein a method for preparing a feedstock material for large-scale production of lactic acid or a salt thereof, the method comprising: (i) providing an unprocessed organic waste; (ii) mixing the unprocessed organic waste with a partially-dried composition of spores of the L-lactic acid producer Bacillus coagulans; and (iii) subjecting the unprocessed organic waste to one or more of mechanical treatment, chemical treatment and enzymatic treatment, wherein the mixing of step (ii) is carried out prior to, or during, the one or more treatments of step (iii), wherein preparing the feedstock material is carried out under non-sterile conditions, and wherein germination of at least a portion of the B. coagulans spores is induced during the preparation of the feedstock material such that the organic waste is partially converted into L-lactic acid, thereby obtaining a feedstock material for large-scale production of lactic acid.

In some embodiments, there is provided herein a method for reducing microorganism load in organic waste and enriching for L-lactic acid prior to large-scale production of L-lactic acid, the method comprising: (i) providing an unprocessed organic waste; (ii) mixing the unprocessed organic waste with a dried composition of spores of the L-lactic acid producer Bacillus coagulans; and (iii) subjecting the unprocessed organic waste to one or more of mechanical treatment, chemical treatment and enzymatic treatment, wherein the mixing of step (ii) is carried out prior to, or during, the one or more treatments of step (iii), wherein the reduction of microorganism load and enrichment for L-lactic acid is carried out without the addition of a pH-adjusting agent and under non-sterile conditions, and wherein germination of at least a portion of the B. coagulans spores is induced during the processing of the organic waste such that the organic waste is partially converted into L-lactic acid, thereby obtaining a decrease of pH reducing the load of microorganisms endogenously present in the organic waste, and an enrichment of L-lactic acid.

In some embodiments, there is provided herein a method for reducing microorganism load in organic waste and enriching for L-lactic acid prior to large-scale production of L-lactic acid, the method comprising: (i) providing an unprocessed organic waste; (ii) mixing the unprocessed organic waste with a partially-dried composition of spores of the L-lactic acid producer Bacillus coagulans; and (iii) subjecting the unprocessed organic waste to one or more of mechanical treatment, chemical treatment and enzymatic treatment, wherein the mixing of step (ii) is carried out prior to, or during, the one or more treatments of step (iii), wherein the reduction of microorganism load and enrichment for L-lactic acid is carried out without the addition of a pH-adjusting agent and under non-sterile conditions, and wherein germination of at least a portion of the B. coagulans spores is induced during the processing of the organic waste such that the organic waste is partially converted into L-lactic acid, thereby obtaining a decrease of pH reducing the load of microorganisms endogenously present in the organic waste, and an enrichment of L-lactic acid.

In additional embodiments, there is provided herein a method for preparing a feedstock material for large-scale production of L-lactic acid or a salt thereof, the method comprising: (i) providing an unprocessed organic waste; (ii) mixing the unprocessed organic waste with a dried or partially-dried composition of spores of the L-lactic acid producer Bacillus coagulans; and (iii) subjecting the unprocessed organic waste to one or more of mechanical treatment, chemical treatment and enzymatic treatment, wherein the mixing of step (ii) is carried out prior to, and/or during, the one or more treatments of step (iii), wherein preparing the feedstock material is carried out under non-sterile conditions, and wherein germination of at least a portion of the B. coagulans spores occurs during the preparation of the feedstock material such that the organic waste is partially converted into L-lactic acid and pH is reduced, thereby obtaining a feedstock material for large-scale production of L-lactic acid.

In some embodiments, there is provided herein a method for preparing a feedstock material enriched with L-lactic acid compared to D-lactic acid as disclosed herein.

As used herein, “unprocessed” organic waste refers to raw organic waste as collected from its various sources, or to organic waste that was subjected to minimal treatment such as mixing and/or addition of water.

Mechanical treatment of the organic waste according to the present invention may include, for example, mixing, separation of impurities such as insoluble impurities (e.g., decanter centrifugation, filtration), shredding, grinding, mincing or a combination thereof.

Chemical treatment may include, for example, thermal treatment, acid treatment or a combination thereof.

Enzymatic treatment may include, for example, saccharification using one or more polysaccharide-degrading enzyme.

In some embodiments, the organic waste is subjected to mechanical treatment and enzymatic treatment.

In some embodiments, the dried or partially-dried composition of spores is reconstituted in a solution, e.g., a lactic acid solution, prior to mixing with the organic waste. Thus, mixing or contacting a dried or partially-dried spore composition with organic waste as disclosed herein encompasses direct inoculation of the spore composition into the organic waste and also inoculation of a reconstituted spore composition.

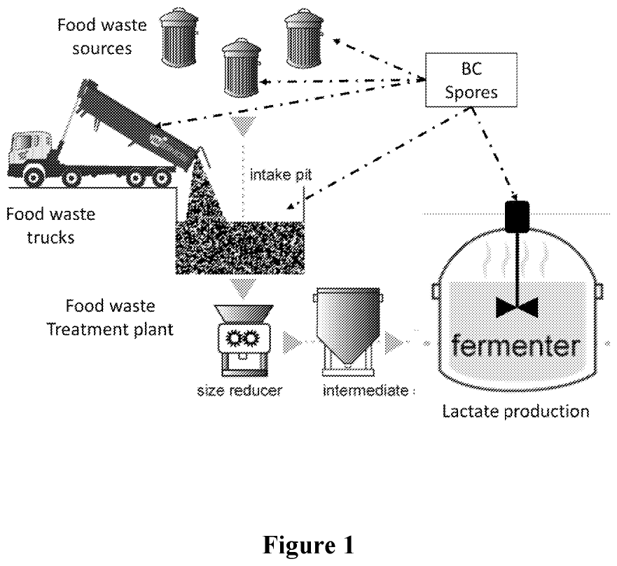

Referring now to the drawings, FIG. 1 illustrates seeding dried or partially-dried compositions of B. coagulans spores according to some embodiments of the present invention. The embodiments illustrated in FIG. 1 pertain to various stages during the handling and pretreatment of organic food waste in which dried or partially-dried compositions of B. coagulans spores can be mixed with the food waste. For example: seeding into food waste sources such as garbage bins, seeding into the containers of trucks during the transportation of the food waste to waste management facilities, seeding into an intake pit of food waste at a waste management facility, seeding during pretreatment of the food waste in food waste treatment plants, for example, seeding into a size reducer or into an intermediate container. Each possibility represents a separate embodiment of the present invention.

The pretreated food waste is transferred into a fermenter for controlled production of lactic acid. “Controlled production” or “controlled fermentation” as used herein refers to lactic acid fermentation that is carried out under controlled conditions, for example: lactic acid fermentation under the control of one or more of the following parameters: temperature, pH, levels of nutrients, agitation rate and aeration (aerobic/anaerobic/microaerophilic conditions). In some embodiments, additional B. coagulans spores are seeded into the fermenter. In other embodiments, no additional B. coagulans spores are seeded, and the fermentation is carried out using B. coagulans spores seeded prior to the controlled production stage. Following the controlled production stage, the fermentation broth is processed to recover the fermentation product, namely, lactic acid or a salt thereof.

In some embodiments, a controlled fermentation according to the present invention is an open fermentation without sterilization, e.g., without chemical-thermo-steam-sterilization of the feedstock before entering the fermenter (using jet cooker etc.) or in the fermenter tank itself. This method of open fermentation saves time and resources that are needed for chemical-thermo-steam-sterilization.

In other embodiments, a controlled fermentation according to the present invention is carried out in a closed fermenter under sterile conditions. According to these embodiments, the pretreated organic waste is subjected to sterilization, e.g., chemical-thermo-steam-sterilization before the controlled production of lactic acid is carried out. According to these embodiments, re-seeding of B. coagulans (e.g., B. coagulans spores) into the sterilized, pretreated organic waste is carried out.

Organic waste management facilities handle collection, transport, processing, recycling/disposal and monitoring of waste materials. In order to recycle the waste into useful chemicals such as lactic acid, namely, utilize the organic waste as a substrate for industrial fermentation processes, an on-site fermentation system is typically required. The conventional method of inoculating industrial fermenters utilizes a wet inoculum of vegetative bacteria (wet seed train). This method has many disadvantages that makes it difficult to implement in waste management facilities, including the need to (i) tightly synchronize the wet seed preparation with the exact inoculation time of the production fermenter, (ii) have an on-site seed train production line which includes a few smaller scale fermenters for the production of the wet seed train (typically a ratio of 1:10 down to few liters flasks).

The wet seed train is a time-consuming and resource-exhausting process. It increases the production time, which consequently limits the number of fermentation cycles that can be performed per a given time period.

According to the present invention, simple integration of lactic acid production into organic waste management facilities is enabled, for on-site production of lactic acid from the organic waste. The compositions of dried or semi-dried spores as disclosed herein can be easily transported to the waste management site, stored and removed from storage upon need.

In some embodiments, a need for seed line is eliminated by the present invention.

Importantly, the preparation of the dried or partially-dried composition can be done at a site that is separated by time and location from the waste management facility.

In addition, the fact that the dried or partially-dried seed can be prepared weeks or months ahead, put in storage and be available immediately for mixing with organic waste, significantly shortens the lactic acid production process.

Lactic acid production from organic waste typically comprises (i) degradation of polysaccharides that are present in the waste using one or more polysaccharide-degrading enzyme in order to release soluble reducing sugars that are suitable for fermentation (“saccharification”); and (ii) fermentation of reducing sugars to lactic acid by a lactic-acid producing microorganism (e.g., Bacillus coagulans as disclosed herein).

Renewable carbohydrate sources for lactic acid production typically include varied ratios of reducing sugars (glucose, fructose, lactose, etc.), but also large amounts of polysaccharides such as starch and optionally also lignocellulosic material. Typically, lactic acid-producing microorganisms can utilize reducing sugars like glucose and fructose, but do not have the ability to degrade polysaccharides like starch and cellulose. Thus, to utilize such polysaccharides the process requires adding polysaccharide-degrading enzymes, optionally in combination with chemical treatment, to degrade the polysaccharides and release reducing sugars. The integration of polysaccharide-degrading enzymes into the process may be sequentially, such that the substrate is treated with one or more polysaccharide-degrading enzymes and subsequently the lactic acid-producing microorganism is added and ferments the reducing sugars, or simultaneously, where the one or more polysaccharide-degrading enzymes and the lactic acid-producing microorganism are mixed together to perform simultaneous saccharification and fermentation. While the simultaneous process reduces the overall time that is required to obtain lactic acid from complex carbohydrate sources, one of its main challenges is the need to match the conditions for both bacterial growth and enzyme activity.

According to some embodiments, the methods of the present invention employ simultaneous saccharification and fermentation. Polysaccharide-degrading enzyme(s) are added to the organic waste together with a dried or partially-dried composition of Bacillus coagulans spores, to obtain simultaneous degradation of polysaccharides present in the waste and production of lactic acid.

When saccharification and fermentation are carried out as separate sequential steps, each step may take between about 18-24 hours. Conducting the two steps simultaneously significantly shortens the process, which results in improved productivity, as more organic waste can be converted to lactic acid per a given time period.

Bacillus coagulans Spore Compositions

Bacillus coagulans is a Gram-positive, thermophilic, facultative anaerobic, spore-forming bacterium that produces lactic acid, particularly L-lactic acid. B. coagulans has been proposed for industrial fermentation processes to produce L-lactic acid. B. coagulans has also been shown to maintain normal intestinal microflora and improve digestibility, and is commonly marketed as a probiotic to maintain the ecological balance of the intestinal microflora and normal gut function. For example, LactoSpore® is a Bacillus coagulans (MTCC 5856) spore preparation intended for use as a probiotic, containing a spray-dried powder of B. coagulans spores mixed with maltodextrin.

Yadav et al. (2009) Indian Journal of Chemical Technology, 16:519-522 examined calcium lactate, calcium gluconate, Spirulina and maltodextrin as probiotic protectants of Bacillus coagulans during spray drying.

Bacillus coagulans strains that may be used according to the present invention include but are not limited to: B. coagulans ATCC 8038 DSM 2312, B. coagulans ATCC 23498 DSM 2314, B. coagulans MTCC 5856, B. coagulans PTA-6086 (GBI-30, 6086), B. coagulans SNZ 1969. Each possibility represents a separate embodiment of the present invention.

In some embodiments of the present invention, the spores of B. coagulans are supplemented by spores originating from at least one additional sporulating L-lactic acid producing bacteria species. The additional sporulating L-lactic acid producing bacteria species may include least one of: Bacillus stearothermophilus, Bacillus licheniformis, Bacillus subtilis, Bacillus laevolacticus, Bacillus racemilacticus, Bacillus thermoamylovorans, Sporolactobacillus, Sporolactobacillus shoreicorticis, Sporolactobacillus vineae, Sporolactobacillus nakayamae. Terrilactibacillus laevilacticus, and Terrilactibacillus tamarindi. Each possibility represents a separate embodiment.

In some further embodiments of the present invention, the spores originating from at least one additional sporulating L-lactic acid producing bacteria species may at least partially replace the spores of B. coagulans, in the process of mixing with the unprocessed organic waste and at least partially converting the waste into L-lactic acid. In some particular embodiments, the spores from the additional bacteria species may be used instead of the spores of B. coagulans.

Spores may be prepared, for example, as follows: in the first step, a pure culture of B. coagulans is inoculated to a sterile seed medium and incubated on shaker at 30-55° C. for example 45-55° C., for 12-24 hours. The seed culture is then transferred to a sporulation medium and incubated at 30-55° C., for example 45-55° C., for 24-48 hours. Induction of sporulation requires stress conditions, for example, lack of nutrients, a relatively rich nitrogen source, such as yeast extract, along with limitation of the carbon and phosphor, presence of Mn²⁺ and Ca²⁺ ions, pH in the range of 5-8 (preferably between 5-7, or 5-6.5), incubation of 24-48 hours (preferably 24 hours), and combinations of the aforementioned stress-inducing factors. The spore concentration in the obtained spore culture is preferably at least 10\hat{ }7 spores/ml, more preferably at least 10\hat{ }8 spores/ml. Each possibility represents a separate embodiment.

Following incubation, the broth is harvested, centrifuged and the pellet is collected. In some embodiments, the harvested pellet, referred to herein as “semi-dried” or “partially-dried” preparation of the spores (moisture content in the range of 15%-30% w/w), is weighed and subsequently mixed with a magnesium lactate solution to obtain a composition comprising the harvested spores and 15-25% magnesium lactate (w/w of the total weight of the composition). In some embodiments, the concentration of magnesium lactate in the composition comprising the harvested spores (prior to drying) is in the range of 15-20% (w/w), for example, 15%, 16%, 17%, 18%, 19% or 20% (w/w) of the total weight of the composition. Each possibility represents a separate embodiment of the present invention. In some embodiments, the composition is dried, for example, spray-dried (e.g. inlet air temperature 180° C. and outlet air temperature 90° C.) or heat-dried at 80° C., to obtain a dried spore composition in a powder form. The moisture content of a dried spore composition according to the present invention is up to 15% (w/w), preferably up to 10% (w/w), typically between 4%-10% w/w. Each possibility represents a separate embodiment of the present invention.

In some embodiments, heat selection at a temperature of 70° C.-80° C. is typically carried out following incubation and prior to drying.

In some embodiments, following drying, a dried composition in a powder form according to the present invention includes at least 10\hat{ }8 spores/g powder, for example, 10\hat{ }8-10\hat{ }10 spores/g powder. In some embodiments, a dried composition according to the present invention includes, for example 10\hat{ }8, 10\hat{ }9, 10\hat{ }10 spores/g powder. Each possibility represents a separate embodiment of the present invention. A dried composition according to the present invention further includes magnesium lactate, at a concentration of 40-60% (w/w), for example, 45%-55% (w/w), 40%-50% (w/w), 50%-60% (w/w). Each possibility represents a separate embodiment of the present invention.

In some embodiments, a dried composition of B. coagulans spores according to the present invention further comprises one or more polysaccharide-degrading enzyme selected from an amylase, a cellulase and a hemicellulase. In some particular embodiments, a dried composition of B. coagulans spores according to the present invention comprises a glucoamylase. In some exemplary embodiments, a dried composition of B. coagulans spores according to the present invention comprises a glucoamylase from Aspergillus niger.

In some embodiments, a dried composition according to the present invention does not require cold storage prior to use thereof. Thus, in some embodiments, a need for cold storage of the lactic-acid producing microbe is eliminated by the methods of the present invention.

According to the present invention, un-immobilized spores are used.

According to embodiments of the present invention, activation of the spores prior to inoculation into the fermenter is not required. For example, heat activation prior to inoculation into the fermenter is not required. As a further example, acid activation is not required prior to, or following, inoculation into the fermenter.

In some embodiments, following contacting with the organic waste substrate as disclosed herein, at least 90% of the spores germinate and produce vegetative cells, for example between 90%-100% of the spores germinate and produce vegetative cells.

Lactic Acid Production from Organic Waste

As used herein, the term “lactic acid” refers to the hydroxycarboxylic acid with the chemical formula CH₃CH(OH)CO₂H. The terms lactic acid or lactate (unprotonated lactic acid) can refer to the stereoisomers of lactic acid: L-lactic acid/L-lactate, D-lactic acid/D-lactate, or to a combination thereof.

For most industrial applications, L-lactic acid monomers with high purity (optical purity) are required in order to produce polylactic acid (PLA) with suitable properties. Thus, the methods and systems of the present invention are directed, in particular, to processes for the production of L-lactic acid or L-lactate salts at high yields.

Organic waste suitable for use according to the present invention is typically a complex organic waste comprising solid and non-solid materials. A complex organic waste includes carbohydrates for fermentation (soluble carbohydrates available for fermentation and/or polysaccharides that need to be decomposed via enzymes to release soluble carbohydrates for fermentation) and further contains impurities such as salts, lipids, proteins, color components, inert materials and more. Examples of organic wastes for use according to the present invention include, but is not limited to, food waste, organic fraction of municipal waste, agricultural waste, plant material, and a mixture or combination thereof. Each possibility represents a separate embodiment. Food waste in accordance with the present invention encompasses food waste of plant origin. Food waste in accordance with the present invention encompasses household food waste, commercial food waste, and industrial food waste. The organic food waste may originate from vegetable and fruit residues, plants, cooked food, protein residues, slaughter waste, and combinations thereof. Industrial organic food waste may include factory waste such as by products, factory rejects, market returns or trimmings of inedible food portions (such as peels). Commercial organic food waste may include waste from shopping malls, restaurants, supermarkets, etc. Plant material in accordance with the present invention encompasses agricultural waste and manmade products such as paper waste. In some embodiments, the organic waste comprises endogenous D-lactic acid, L-lactic acid or both L- and D-lactic acid, originating, for example, from natural fermentation processes, e.g., in dairy products.

Organic waste for use with the methods and systems of the present invention typically comprises complex polysaccharides including starch, cellulose, hemicellulose, and combinations thereof. The organic waste also comprises soluble reducing sugars, and/or is saccharified with one or more polysaccharide-degrading enzyme to obtain soluble reducing sugars (fermentable carbohydrates). As used herein, the term “fermentable carbohydrates” refers to carbohydrates which can be fermented by Bacillus coagulans to lactic acid during a fermentation process. The reducing sugars typically comprise C5 sugars (pentoses), C6 sugars (hexoses) or a combination thereof. In some embodiments, said reducing sugars comprise glucose. In some embodiments, said reducing sugars comprise xylan.

Organic waste according to the present invention typically comprises complex polysaccharides and reducing sugars at varying ratios. The composition depends on the source of the waste, where some organic wastes may be more starch-rich (e.g., food waste from bakeries, mixed food waste of municipalities) and others may be rich with lignocellulosic material (e.g., agricultural waste). In some embodiments, the organic waste includes a combination of wastes from different sources.

In some embodiments, the percentage of at least one of starch, cellulose and hemicellulose in the organic waste is determined prior to treatment with one or more polysaccharide-degrading enzyme. In some embodiments, the percentage of soluble reducing sugars is determined prior to the fermentation.

Organic waste typically includes nitrogen sources and other nutrients needed for bacterial growth and lactic acid production, but such nutrients may also be supplied separately to the lactic acid production fermenter if needed.

Pretreatment of the organic waste according to the present invention typically includes decreasing particle size and increasing surface area. In some embodiments, pretreatment includes inactivating endogenous bacteria within the waste. In some embodiments, the pretreatment comprises shredding and mincing. In some embodiments, the pretreatment comprises shredding, mincing and sterilization.

Sterilization may be carried out by methods known in the art, including for example, high pressure steam, chemical sterilization, cooking at boiling point, UV radiation or sonication.

The pretreatment may include, for example, shredding and sterilization. Pretreatment may also include mincing with an equal amount of water using a waste mincer, such as, e.g., an extruder, sonicator, shredder or blender.

In some embodiments, one or more saccharide-degrading enzyme and a dried or partially-dried composition of B. coagulans spores are added simultaneously to a fermentation reactor containing a pretreated organic waste. In additional embodiments, the time period between the addition of one or more saccharide-degrading enzyme and the addition of a dried or partially-dried composition of B. coagulans spores is in the range of 0-5 hours, including each value within the range. In other embodiments, one or more saccharide-degrading enzyme is added to the fermenter 1-5 hours after a dried or partially-dried composition of B. coagulans spores is added, for example, 1 hour, at least 2 hours, 2 hours, 3 hours, 4 hours or 5 hours after a dried or partially-dried composition of B. coagulans spores is added. Each possibility represents a separate embodiment. In other embodiments, one or more saccharide-degrading enzyme is added to the fermenter before the dried or partially-dried composition of B. coagulans spores is added.

As used herein, “mixing a dried composition of B. coagulans spores in a fermentation reactor”, “adding a dried composition of B. coagulans spores to fermentation reactor” and the like encompass adding the dried powder directly into the fermentation reactor, or reconstituting the powder in a reconstitution medium. The present invention particularly discloses reconstitution in a lactic acid solution, to achieve both reconstitution and inhibition of microbial contaminants that may be present.

Large-scale lactic acid fermentation according to the present invention is typically carried out under anaerobic or microaerophilic conditions, using batch, fed-batch, continuous or semi-continuous fermentation. Each possibility represents a separate embodiment of the present invention.

In batch fermentation, the carbon substrates and other components are loaded into the reactor, and, when the fermentation is completed, the product is collected. Except for an alkaline compound for pH control, other ingredients are not added to the reaction before it is completed. The fermentation is kept at substantially constant temperature and pH, where the pH is maintained by adding the alkaline compound.

In fed-batch fermentation, the substrate is fed continuously or sequentially to the reactor without the removal of fermentation broth (i.e., the product(s) remain in the reactor until the end of the run). Common feeding methods include intermittent, constant, pulse-feeding and exponential feeding.

In continuous fermentation, the substrate is added to the reactor continuously at a fixed rate, and the fermentation products are taken out continuously.

In semi-continuous processes, a portion of the culture is withdrawn at intervals and fresh medium is added to the system. Repeated fed-batch culture, which can be maintained indefinitely, is another name of the semi-continuous process.

Fermentations that produce acidic products such as organic acids etc. are typically performed in the presence of an alkaline compound, such as a metal oxide, a carbonate or a hydroxide. The alkaline compound is added to adjust the pH of the fermentation broth to a desired value, typically in the range of 4-7, including each value within the specified range. The alkaline compound further results in the neutralization of the L-lactic acid to a lactate salt. During fermentation the pH in the fermenter decreases due to the production of the lactic acid, which adversely affects the productivity of the Bacillus coagulans. Adding bases such as magnesium-hydroxide/oxide, sodium-hydroxide, potassium-hydroxide, or calcium-hydroxide adjusts the pH by neutralizing the lactic acid thereby resulting in the formation of a lactate salt.

In some particular embodiments, the present invention recycles organic waste to produce magnesium lactate. In some embodiments, such a process utilizes magnesium hydroxide as the alkaline compound for adjusting pH during fermentation. The fermentation results in lactate monomers and Mg²⁺ ions, that can be recovered as magnesium lactate.

Lactic acid fermentation is typically carried out for about 1-4 days or any amount therebetween, for example, 1-2 days, or 2-4 days, or 3-4 days, including each value within the specified ranges.

After fermentation is completed, the broth may be clarified by centrifugation or passed through a filter press to separate solid residue from the fermented liquid. The filtrate may be concentrated, e.g., using a rotary vacuum evaporator.

The fermentation broth according to the present invention may contain D-lactic acid originating from the organic waste. The D-LA is undesired in the production of L-LA for polymerization as it results in the formation of more D,D-lactide and meso-lactide, which adversely impact the quality of the PLLA final product. In some embodiments, the methods and systems of the present invention advantageously eliminate D-lactic acid by employing a D-lactic acid degrading enzyme or a D-lactic acid utilizing microorganism to the organic waste prior to lactic acid production, or to the fermentation broth during and/or following fermentation. Each possibility represents a separate embodiment.

Currently preferred is the use of a D-lactate oxidase as a D-lactic acid degrading enzyme. A D-lactate oxidase is an enzyme that catalyzes the oxidation of D-lactate to pyruvate and H₂O₂ using O₂ as an electron acceptor. The enzyme uses flavin adenine dinucleotide (FAD) as a co-factor for its catalytic activity. A D-lactate oxidase according to the present invention is typically a soluble D-lactate oxidase (rather than membrane-bound). Advantageously, the enzyme works directly in organic wastes and also in fermentation broths, to eliminate the D-lactic acid. In some embodiments, the D-lactate oxidase is from Gluconobacter sp. In some embodiments, the D-lactate oxidase is from Gluconobacter oxydans (see, for example, GenBank accession number: AAW61807). Elimination of D-lactate from fermentation broths derived from organic wastes using a D-lactate oxidase is described in WO 2020/208635 assigned to the Applicant of the present invention.

Suitable D-lactic acid-utilizing microorganisms within the scope of the present invention include, but are not limited to, an Escherichia coli lacking all three L-lactate dehydrogenases.

As used herein, “elimination”, when referring to D-lactic acid/D-lactate, refers to reduction to residual amounts such that there is no interference with downstream processes of producing L-lactic acid and subsequently polymerization to poly(L-lactic acid) that is suitable for industrial applications. “Residual amounts” indicates less than 1% (w/w) D-lactate, and even more preferably less than 0.5% (w/w) D-lactate, out of the total lactate (L+D) in a treated mixture of a fermentation broth at the end of fermentation. In some particular embodiments, elimination of D-lactate is reduction to less than 0.5% (w/w) D-lactic acid out of the total lactate in a fermentation broth at the end of fermentation.

According to further aspects and embodiments, L-lactate monomers are further purified. The L-lactate monomers may be purified as L-lactate salts. Alternatively, a reacidification step with, e.g., sulfuric acid, may be carried out in order to obtain crude L-lactic acid, followed by purification steps to obtain a purified L-lactic acid.

The purification processes may include distillation, extraction, electrodialysis, adsorption, ion-exchange, crystallization, and combinations of these methods. Several methods are reviewed, for example, in Ghaffar et al. (2014) Journal of Radiation Research and Applied Sciences, 7 (2): 222-229); and López-Garzón et al. (2014) Biotechnol Adv., 32 (5): 873-904). Alternatively, recovery and conversion of lactic acid to lactide in a single step may be used (Dusselier et al. (2015) Science, 349 (6243): 78-80).

In some particular embodiments of the present invention, the alkaline compound used for pH adjustment during fermentation is magnesium hydroxide (Mg(OH)₂), resulting in a fermentation broth comprising lactate monomers and Mg²⁺, which can be recovered as magnesium lactate. A particular downstream purification process for purifying magnesium lactate via crystallization is described in WO 2020/110108, assigned to the Applicant of the present invention. The purification process can be applied to the fermentation broth after treatment that eliminates D-lactate monomers where applicable.

### Saccharide-Degrading Enzymes

“Saccharide-degrading enzymes” as used herein refers to hydrolytic enzymes (or enzymatically-active portions thereof) that catalyze the breakdown of saccharides, including bi-saccharides (di-saccharides), oligosaccharides, polysaccharides and glycoconjugates. Saccharide-degrading enzymes may be selected from the group consisting of glycoside hydrolases, polysaccharide lyases and carbohydrate esterases. Each possibility represents a separate embodiment of the present invention. The saccharide-degrading enzymes for use with the present invention are selected from those that are active towards saccharides (such as polysaccharides) found in organic wastes, including food waste and plant material. In some embodiments, the saccharide-degrading enzymes may be modified enzymes (i.e., enzymes that have been modified and are different from their corresponding wild-type enzymes). In some embodiments, the modification may include one or more mutations that result in improved activity of the enzyme. In some embodiments, the saccharide-degrading enzymes are wild type (WT) enzymes.

The broad group of saccharide-degrading enzymes is divided into enzyme classes and further into enzyme families according to a standard classification system (Cantarel et al. 2009 Nucleic Acids Res 37: D233-238). An informative and updated classification of such enzymes is available on the Carbohydrate-Active Enzymes (CAZy) server (www.cazy.org).

In some embodiments, the saccharide-degrading enzymes used in the present invention are polysaccharide-degrading enzymes. In some embodiments, the polysaccharide-degrading enzymes are enzymes that degrade polysaccharides selected from starch and non-starch plant polysaccharides.

In some embodiments, the polysaccharide-degrading enzymes are glycoside hydrolases.

In some embodiments, the polysaccharide-degrading enzymes are selected from amylases, cellulases and hemicellulases. Each possibility represents a separate embodiment of the present invention.

A cellulase may be selected from, but not limited to: endo-(1,4)-D-glucanase, εχo-(1,4)-β-\ddot{υ}-glucanase, β-glucosidases, Carboxymethylcellulase (CMCase); endoglucanase; cellobiohydrolase; avicelase, celludextrinase, cellulase A, cellulosin AP, alkali cellulase, and pancellase SS. Each possibility is a separate embodiment.

A hemicellulase may be a xylanase. Non-limiting examples of additional hemicellulases include arabinofuranosidases, acetyl esterases, mannanases, a-D-glucuronidases, β-xylosidases, β-mannosidases, β-glucosidases, acetyl-mannanesterases, a-galactosidases, -a-Larabinanases, and β-galactosidases. Each possibility represents a separate embodiment of the present invention.

An amylase may be selected from, but not limited to: glucoamylase, a-amylase; (1,4-a-D-glucan glucanohydrolase; glycogenase) β-Amylase; (1,4-a-D-glucan maltohydrolase; glycogenase; saccharogen amylase) γ-Amylase; (Glucan 1,4-a-glucosidase; amyloglucosidase; Exo-1,4-a-glucosidase; lysosomal a-glucosidase and 1,4-a-D-glucan glucohydrolase. Each possibility is a separate embodiment.

In some embodiments, the saccharide-degrading enzymes used in the present invention are disaccharide-degrading enzymes. In some embodiments, the disaccharide-degrading enzymes are selected from lactases and invertases. Each possibility represents a separate embodiment of the present invention.

The saccharide-degrading enzymes according to the present invention may be from a bacterial source. In some embodiments, the bacterial source is a thermophilic bacterium. The term “thermophilic bacterium” as used herein indicates a bacterium that thrives at temperatures higher than about 45° C., preferably above 50° C. Typically, thermophilic bacteria according to the present invention have optimum growth temperature of between about 45° C. to about 75° C., preferably about 50-70° C. Non-limiting examples of thermophilic bacterial sources for saccharide-degrading enzymes include: Cellulases and hemicellulases—Clostridium sp. (e.g. Clostridium thermocellum), Paenibacillus sp., Thermobifida fusca; Amylases—Bacillus sp. (e.g. Bacillus stearothermophilus), Geobacillus sp. (e.g. Geobacillus thermoleovorans), Chromohalobacter sp., Rhodothermus marinus. Each possibility is a separate embodiment.

In additional embodiments, the bacterial source of the saccharide-degrading enzymes is a mesophilic bacterium. The term “mesophilic bacterium” as used herein indicates a bacterium that thrives at temperatures between about 20° C. and 45° C. Non-limiting examples of mesophilic bacterial sources for saccharide-degrading enzymes include: Cellulases and hemicellulases—Klebsiella sp. (e.g. Klebsiella pneumonia), Cohnel sp., Streptomyces sp, Acetivibrio cellulolyticus, Ruminococcus albus; Amylases-Bacillus sp. (e.g. Bacillus amyloliquefaciens, Bacillus subtilis, Bacillus licheniformis), Lactobacillus fermentum. A person of skill in the art understands that some mesophilic bacteria (e.g., several Bacillus sp.) produce thermostable enzymes.

The saccharide-degrading enzymes according to the present invention may also be from a fungal source. Non-limiting examples of fungal sources for saccharide-degrading enzymes include: Cellulases and hemicellulases—Trichoderma reesei, Humicola insolens, Fusarium oxysporum; Amylases (e.g., glucoamylases)—Aspergillus niger Aspergillus oryzae, Penicillium fellutanum, Thermomyces lanuginosu.

Additional sources for saccharide-degrading enzymes for use in accordance with the present invention can be found, for example, at the CAZy server mentioned above.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Reconstitution of a Dried Spore Composition in a Lactic Acid Solution

A dried composition of B. coagulans spores was suspended in an aqueous solution of 3% lactic acid, 3.47% magnesium lactate, pH 3.5±0.2. For comparison, a dried composition of B. coagulans spores was suspended in water. Both suspensions were incubated at room temperature with stirring for 45 minutes, and subsequently plated on YPD agar plates and grown for 14 hours at 52° C. Total bacteria count (CFU) and spore count (sCFU) were performed after the incubation. The results are summarized in Table 1.

TABLE 1
Spore germination following incubation in a lactic acid solution

| Diluent | CFU | sCFU |
|---|---|---|
| Water | 3*10\hat{ }8 | 1*10\hat{ }8 |
| pH 3.5 Buffer 3% LA | 4*10\hat{ }8 | 2*10\hat{ }8 |

The results showed that B. coagulans spores survived incubation in the lactic acid solution and successfully germinated following such treatment. No substantial differences were observed in the number of bacterial cells that germinated from the spores between water and the lactic acid solution.

### Example 2

Reconstitution of a Dried Spore Composition in a Lactic Acid Solution and Seeding into Sterile or Non-Sterile Organic Waste

A dried composition of B. coagulans spores was suspended in an aqueous solution of 3% lactic acid, 3.47% magnesium lactate, pH 3.5±0.2 as described above. Following suspension, the spores were seeded into a food waste that was subjected to sterilization or into a non-sterile food waste and grown for 14 hours at 52° C. Table 2 shows bacterial cell count in each medium. As can be seen in the table, no substantial differences were observed in the number of bacterial cells that germinated from the spores in sterile and non-sterile food waste. In addition, production of lactic acid was observed in both cases.

TABLE 2
Spore germination in sterile and non-sterile food waste

| | | Bacterial | Bacterial |
|---|---|---|---|
| | | count before | count after |
| | | seeding | seeding (1:100 |
| Diluent | Inoculation to | (CFU) | dilution) (CFU) |
| pH 3.5 Buffer 3% | Sterile food waste | 3.2*10\hat{ }8 | 1.0*10\hat{ }6 |
| LA | | | |
| pH 3.5 Buffer 3% | Food waste without | 1.4*10\hat{ }8 | 3.9*10\hat{ }6 |
| LA | sterilization | | |

### Example 3

Acidification of Media by Germinating Spores of B. coagulans Under Various Conditions
A. 10\hat{ }4-10\hat{ }8 spores/ml were inoculated into 10 ml of food waste in 50 ml Falcon® tubes. The pH of the waste was adjusted to pH-7 prior to inoculation. The tubes were kept at room temperature or incubated at 37° C. and 52° C. pH was measured after 17 hours and was found to be between 4.5 and 5.5.
B. 10\hat{ }4-10\hat{ }8 spores/ml were inoculated into 10 ml of sterile medium comprised of 10 g/l soy peptone, 5 g/l yeast extract and 10 g/l glucose. Medium pH was 6.4-6.9. The Falcon® tube was kept at room temperature or incubated at 37° C. and 52° C. pH was measured after 10 hours and was found to be between 4.5 and 5.5.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for preparing a feedstock material for large-scale production of L-lactic acid or a salt thereof, the method comprising:
(i) providing an unprocessed organic waste;
(ii) mixing the unprocessed organic waste with a dried or partially-dried composition of spores of a spore-forming L-lactic acid producing Bacillus sp.; and
(iii) subjecting the unprocessed organic waste to one or more of mechanical treatment, chemical treatment and enzymatic treatment;
wherein the mixing of step (ii) is carried out prior to, and/or during, the one or more treatments of step (iii),
wherein preparing the feedstock material is carried out under non-sterile conditions, and
wherein germination of at least a portion of the Bacillus spores occurs during the preparation of the feedstock material such that the organic waste is partially converted into L-lactic acid,
thereby obtaining a feedstock material enriched with L-lactic acid for large-scale production of L-lactic acid.

2. The method of claim 1, wherein the organic waste is selected from the group consisting of food waste, municipal waste, agricultural waste, plant material and a mixture or combination thereof.

3. The method of claim 2, wherein the organic waste is selected from a solid organic waste, a semi-solid organic waste with a water content in the range of 30%-95% (w/w), and a liquid organic waste.

4-5. (canceled)

6. The method of claim 1, wherein the mixing in step (ii) is carried out in one or more of: organic waste collection containers, transport vehicles that transport organic waste to waste management facilities, and organic waste management facility prior to, or during, the one or more treatments of step (iii).

7-8. (canceled)

9. The method of claim 1, wherein the one or more treatments of step (iii) comprise size reduction by mincing, shredding, grinding or a combination thereof.

10. The method of claim 1, wherein the one or more treatments of step (iii) comprise saccharification using one or more polysaccharide-degrading enzyme.

11. The method of claim 1, wherein step (ii) further comprises mixing with one or more polysaccharide-degrading enzyme.

12. A method for reducing microorganism load in organic waste and enriching for L-lactic acid prior to large-scale production of L-lactic acid, the method comprising:
(i) providing an unprocessed organic waste;
(ii) mixing the unprocessed organic waste with a dried or partially-dried composition of spores of a spore-forming L-lactic acid producing Bacillus sp.; and
(iii) subjecting the unprocessed organic waste to one or more of mechanical treatment, chemical treatment and enzymatic treatment;
wherein the mixing of step (ii) is carried out prior to, and/or during, the one or more treatments of step (iii),
wherein the reduction of microorganism load and enrichment for L-lactic acid is carried out without the addition of a pH-adjusting agent and under non-sterile conditions, and
wherein germination of at least a portion of the Bacillus spores occurs during the processing of the organic waste such that the organic waste is partially converted into L-lactic acid,
thereby obtaining a decrease of pH reducing the load of microorganisms endogenously present in the organic waste, and an enrichment of L-lactic acid.

13. A method for large-scale production of L-lactic acid, the method comprising:
(a) preparing a feedstock material according to claim 1;
(b) optionally mixing the feedstock material with additional Bacillus spores; and
(c) incubating the feedstock material in a fermentation reactor under controlled conditions for lactic acid production by the Bacillus sp., to thereby produce L-lactic acid.

14. The method of claim 13, wherein the feedstock is not sterilized prior to the incubation in the fermentation reactor, and the large-scale production is an open-fermentation carried out under non-sterile conditions.

15. The method of claim 13, wherein the feedstock is sterilized prior to the incubation in the fermentation reactor, and the method further comprises mixing the feedstock material with additional Bacillus spores following sterilization.

16. The method of claim 13, wherein step (c) comprises incubating the feedstock material in a fermentation reactor with one or more polysaccharide-degrading enzyme under controlled conditions for degradation of polysaccharides by the one or more polysaccharide-degrading enzyme and production of lactic acid by the Bacillus sp.

17-19. (canceled)

20. A feedstock material for large-scale production of L-lactic acid, comprising:
(a) semi-solid processed organic waste comprising 5%-30% solids (w/w/); and
(b) B. coagulans bacterial cells, B. coagulans spores or a combination thereof,
wherein the feedstock product is enriched with L-lactic acid compared to D-lactic acid, and
wherein the pH of the feedstock product is in the range of 3-6.

21. The feedstock material of claim 20, wherein the pH of the feedstock product is in the range of 4-5.

22-34. (canceled)

35. The method of claim 1, wherein the spore-forming L-lactic acid producing Bacillus sp. is Bacillus coagulans.

36. The method of claim 1, wherein the composition of Bacillus spores is a dried composition characterized by moisture content of less or equal to 10% w/w.

37. The method of claim 36, wherein the composition of Bacillus spores is a dried composition comprising magnesium lactate.

38. The method of claim 37, wherein the dried composition of Bacillus spores comprises 10\hat{ }8-10\hat{ }10 spores/g powder, and wherein the concentration of the magnesium lactate in the dried composition is in the range of 40-60% (w/w).

39. The method of claim 12, wherein the spore-forming L-lactic acid producing Bacillus sp. is Bacillus coagulans.

40. The method of claim 13, wherein the spore-forming L-lactic acid producing Bacillus sp. is Bacillus coagulans.
